(19) United States
(12) Patent Application Publication
(10) Pub. No.: US 2012/0330350 A1
(43) Pub. Date: Dec. 27, 2012

(54) METHODS AND SYSTEMS FOR PERFORMING THROMBECTOMY PROCEDURES

(76) Inventors: Donald K. Jones, Dripping Springs, TX (US); Vladimir Mitelberg, Austin, TX (US)

(21) Appl. No.: 13/533,859

(22) Filed: Jun. 26, 2012

Related U.S. Application Data
(60) Provisional application No. 61/501,691, filed on Jun. 27, 2011.
(60) Provisional application No. 61/501,729, filed on Jun. 27, 2011.

(51) Int. Cl. A61F 2/01

(52) U.S. Cl. 606/200

(57) ABSTRACT

The present invention relates to methods and systems for performing intralumenal procedures including revascularization and removal of foreign objects from a body lumen. More particularly the present invention relates to systems utilizing thrombectomy devices and methods of performing medical procedures to remove thrombus, emboli, foreign objects and or re-establish the intravascular flow of blood.

### CROSS-REFERENCE TO RELATED APPLICATIONS

This application claims the benefit of U.S. Prov. Ser. No. 61/501,691 filed Jun. 27, 2011 and U.S. Prov. Ser. No. 61/501,729 filed Jun. 27, 2011, all of which are hereby incorporated by reference herein in their entireties.

### BACKGROUND OF THE INVENTION

The field of intralumenal therapy for the treatment of vascular disease states has for many years focused on the use of many different types of therapeutic devices. While it is currently unforeseeable that one particular device will be suitable to treat all types of vascular disease states it may however be possible to reduce the number of devices used for some disease states while at the same time improve patient outcomes at a reduced cost. To identify potential opportunities to improve the efficiency and efficacy of the devices and procedures it is important for one to understand the state of the art relative to some of the more common disease states.

One cerebrovascular disease state is ischemia resulting from reduced or blocked arterial blood flow. The arterial blockage may be due to thrombus, plaque, foreign objects or a combination thereof. Generally, soft thrombus created elsewhere in the body (for example due to atrial fibrillation) that lodges in the distal cerebrovasculature may be disrupted or dissolved using mechanical devices and or thrombolytic drugs. While guidewires are typically used to disrupt the thrombus, some sophisticated thrombectomy devices have been proposed. For instance U.S. Pat. No. 4,762,130 to Fogarty et al., entitled, “Catheter with Corkscrew-Like Balloon”, U.S. Pat. No. 4,998,919 of Schepp-Pesh et al., entitled, “Thrombectomy Apparatus”, U.S. Pat. No. 5,417,703 to Brown et al., entitled “Thrombectomy Devices and Methods of Using Same”, and U.S. Pat. No. 6,663,650 to Sepetka et al., entitiled, “Systems, Methods and Devices for Removing Obstructions from a Blood Vessel” discloses devices such as catheter based corkscrew balloons, baskets or filter wires and helical coiled retrievers. Commercial and prototype versions of these devices have shown only marginal improvements over guidewires due to an inability to adequately grasp the thrombus or to gain vascular access distal to the thrombus (i.e. distal advancement of the device pushes the thrombus distally).

Plaque buildup within the lumen of the vessel, known as atherosclerotic disease, is not generally responsive to thrombolytics or mechanical disruption using guidewires. The approach to the treatment of neurovascular atherosclerotic disease has been to use modified technology developed for the treatment of cardiovascular atherosclerotic disease, such as balloons and stents, to expand the vessel at the site of the lesion to re-establish blood flow. For instance, U.S. Pat. No. 4,768,507 to Fischell et al., entitled, “Intravascular Stent and Percutaneous Insertion Catheter System for the Dilation of an Arterial Stenosis and the Prevention of Arterial Restenosis” discloses a system used for placing a coil spring stent into a vessel for the purposes of enhancing luminal dilation, preventing arterial restenosis and preventing vessel blockage resulting from intimal dissection following balloon and other methods of angioplasty. The coil spring stent is placed into spiral grooves on an insertion catheter. A back groove of the insertion catheter contains the most proximal coil of the coil spring stent which is prevented from springing radially outward by a flange. The coil spring stent is deployed when an outer cylinder is moved proximally allowing the stent to expand. Other stent systems include those disclosed in U.S. Pat. No. 4,512,338 to Balko, et al., entitled, “Process for Restoring Patency to Body Vessels”, U.S. Pat. No. 5,354,309 to Schnepp Pesch et al., entitled, “Apparatus for Widening a Body Cavity” and U.S. Pat. No. 6,833,003 to Jones et al., entitled, “Expandable Stent and Delivery System”. While the aforementioned devices may have the ability to access the cerebrovasculature, they lack sufficient structural coverage of the lesion to achieve the desired patency of the vessel without the use of a balloon device.

### SUMMARY OF THE INVENTION

In accordance with one aspect of the present invention there is provided a medical device system for restoring patency of a body lumen in a mammal. The thrombectomy system includes a thrombectomy catheter having a proximal hub assembly and a distal end, a longitudinally extending balloon extending distal to the catheter distal end, an expandable retrieval assembly positioned distal to the balloon and an inflation source member coupled to the proximal hub. The proximal end of the extendable balloon member is coupled to the distal end of the catheter and the expandable retrieval assembly is coupled to the distal end of the balloon member. An elongate tether member is positioned within the catheter lumen and preferably coupled to the balloon member distal end and proximal end of the retrieval assembly. The tether member extends proximally through the thrombectomy catheter lumen and proximal to the proximal end of the hub assembly. The expandable retrieval assembly comprises a capture member that generally takes the form of a “closed ended” framework resembling a basket where the distal end is closed with struts or otherwise designed to retain thrombus. The proximal end of the framework is “open” and has a diameter commensurate with the inner diameter of the target vessel for receiving a thrombus. The capture member of the retrieval assembly is formed of a resilient material and has a biased expanded configuration such that the capture member may be constrained to a smaller diameter when positioned in the catheter lumen for delivery and when deployed from the catheter lumen and unconstrained, return to an expanded configuration. For delivery to a desired target site, the longitudinally extending balloon is everted and positioned within the lumen of the thrombectomy catheter such that the distal end of the balloon is proximal to the distal end of the catheter. The retrieval assembly is compressed and positioned within the balloon within the catheter lumen. The balloon member of the delivery catheter is typically formed of a thin walled polymeric tube in which the distal end of the tube has been sealed and the proximal end of the balloon member is coupled to the distal end of the catheter such that the lumen of the catheter is in fluid communication with the interior surface of the balloon. The balloon member is preferably formed of a high strength non-compliant polymeric material such as nylon, polyester and others, however, metallic materials such as thin-film nitinol or other alloys may also be suitable. The inflation source member is coupled to the proximal end of the catheter and used to apply fluid pressure to the lumen of catheter at a level sufficient to cause the balloon member to extend longitudinally from the catheter lumen, thus deploying the retrieval assembly. The preferred fluids include liquids such as saline although gases such as carbon dioxide gas may be suitable for some system configurations. The amount of fluid pressure required to inflate the balloon member is in part related to the increased friction force between the balloon member inner surface and the interior wall of the catheter lumen due to the outward force applied by the constrained collapsed retrieval assembly. The inflation source member preferably takes the form of a syringe (threaded or non-threaded), however other inflation sources such as a pressurized fluid source having a valve assembly or a controllable fluid delivery pump are also suitable.

In accordance with another aspect of the present invention there is provided a thrombectomy system retrieval assembly comprising biocompatible resilient materials. Suitable resilient materials include metal alloys such as nitinol, titanium, stainless steel and cobalt chromium and any alloys thereof Additional suitable materials include polymers such as polyimides, polyamides, fluoropolymers, polyetheretherketone (PEEK) and shape memory polymers. These materials may be formed into desired shapes by a variety of methods which are appropriate to the materials be in utilized such as laser cutting, injection molding, welding, electrochemical machining, machining, photo-etching and casting.

In accordance with yet another aspect of the present invention there is provided an expandable retrieval assembly that includes a mesh coupled to a capture member framework. Alternatively the retrieval assembly may be formed as an expandable framework and include a mesh covering.

In accordance with still another aspect of the present invention there is provided an expandable retrieval assembly that includes a capture member that takes the form of a coil having multiple winds or turns. The coil may have a conical or tapering shape. The coil may also include a plurality of side extension members extending outwardly from a coil wind in a plane generally defined by adjacent winds or turns. The side extension members extending from one turn of the coil may overlap an adjacent turn or overlap the side extension members of an adjacent turn.

In accordance with another aspect of the present invention there is provided an expandable retrieval assembly having a generally helical backbone and side extension members which may take various configurations comprising any of the following: side extension members on each side of the backbone which are uniformly spaced along the length of the backbone; side extension members on each side of the backbone which are not uniformly spaced along the length of the backbone; side extension members having a curved shape; side extension members having a straight shape; side extension members extending from the backbone in an angled direction; side extension members having different lengths; side extension members having apertures; side extension members having radio-opaque markers; backbones having apertures; backbones having radio-opaque marker(s).

### BRIEF DESCRIPTION OF THE DRAWINGS

FIG. 1 is a partial cross-sectional view of a thrombectomy system according to an embodiment of the present invention.

FIG. 2A is an enlarged partial cross-sectional view of the distal end of the thrombectomy system according to an embodiment of the present invention.

FIG. 2B is an enlarged partial cross-sectional view of the distal end of an alternate thrombectomy system according to another embodiment of the present invention.

FIG. 3A is a partial cross sectional view of a deployed thrombectomy system according to an embodiment of the present invention.

FIG. 3B is a partial cross sectional view of a deployed thrombectomy system according to another embodiment of the present invention.

FIG. 3C is a partial cross sectional view of a deployed thrombectomy system according to yet another embodiment of the present invention.

FIG. 3D is a partial cross sectional view of a deployed thrombectomy system according to still yet another embodiment of the present invention.

FIGS. 4A through 4F partial cross-sectional views illustrating a method of delivering and deploying a thrombectomy system within a vessel at a target site according to an embodiment of the present invention.

### DETAILED DESCRIPTION OF THE INVENTION

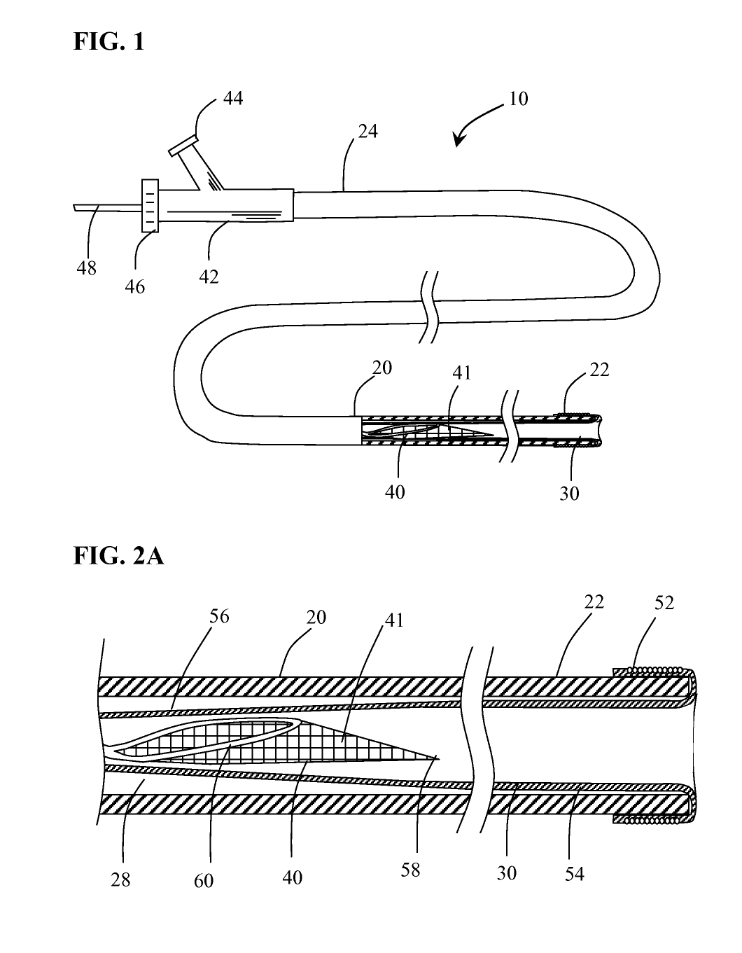

Methods and systems for capturing and removing an embolus or thrombus from an area of the body are herein described. While the terms “thrombectomy” and “thrombus” generally refer to removal of a specific type of embolus, the usage herein should be considered more broadly to include the removal additional types of emboli such as plaque, solid tissue fragments, clots and foreign objects that may block or restrict the normal flow of blood within the vasculature. In other nonvascular lumens within the body, the term “embolus” is herein construed more broadly, to include obstructions of a lumen such as “stones” lodged in a duct. FIG. 1 illustrates a thrombectomy system 10 according to an embodiment of the present invention. Thrombectomy system 10 includes an elongate catheter 20 having a distal end 22, a proximal end 24 and a lumen 28 extending therethrough. Coupled to distal end 22 of catheter 20 is balloon member 30. Balloon member 30 has a delivery configuration in which it is everted and positioned within lumen 28 of catheter 20 at distal end 22. Thrombectomy system 10 also includes collapsible retrieval assembly 40 having flexible mesh 41 for engaging thrombus within a vessel. Retrieval assembly 40 has a delivery configuration in which it is collapsed and positioned within lumen 28 of catheter 20 at distal end 22. Proximal end 24 of catheter 20 is coupled to hub member 42 which includes inflation port 44 and a sealable valve 46. An elongate flexible tether member 48 coupled to balloon member 30 is slidably positioned within lumen 28 and extends through valve 46.

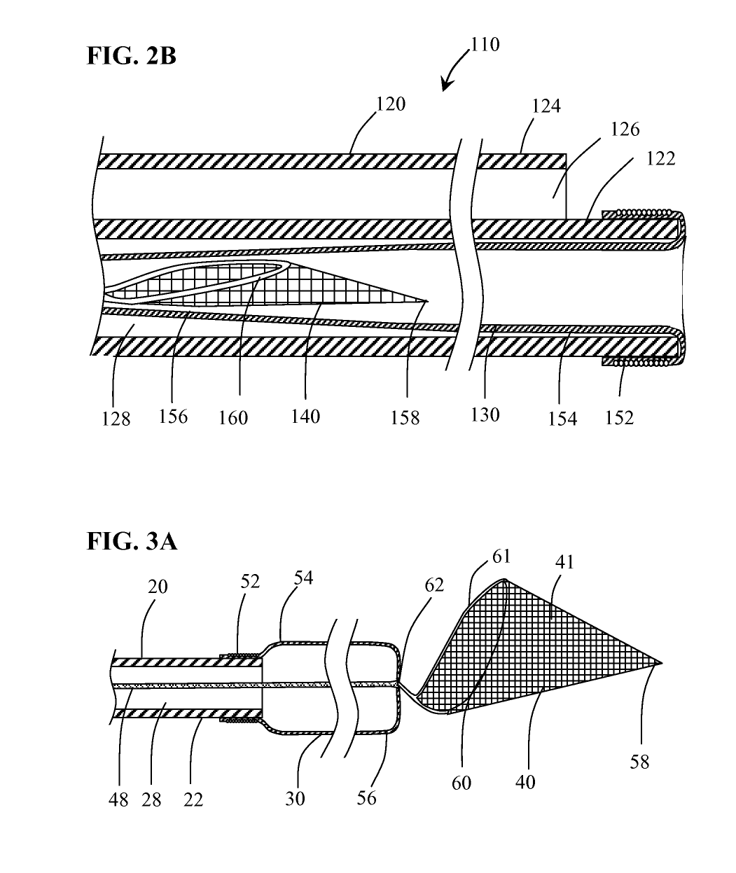

A partial cross sectional view of distal end 22 of catheter 20 is shown in FIG. 2A. While not shown, the construction of catheter 20 may utilize known catheter technologies that incorporate braiding and or coiling using metallic or non-metallic reinforcing filamentous materials to provide high strength while maintaining catheter flexibility. The incorporation of lubricious hydrophilic and or hydrophobic materials on the inner and or outer surface of the catheter is considered to be within the scope of known catheter construction techniques and suitable for use in a thrombectomy system according to embodiments of the present invention. Retaining member 52 is used to affix proximal end 54 of balloon member 30 to catheter distal end 22. The inner diameter of balloon member proximal end 54 is slightly larger than the outer diameter of catheter distal end 22 thereby allowing distal end 22 to be inserted within proximal end 54. Retaining member 52 is shown as a flexible filament (preferably polymeric) wound around proximal end 54 and catheter distal end 22 securing balloon member 30 to catheter 20. Balloon member proximal end 54 and distal end 22 may be secured using other means such as heat fusing, multifilament winds, ultrasonic welding and or gluing to insure a good bond and seal. The distal end 56 of balloon member 30 is completed sealed using any of the aforementioned techniques and positioned everted within catheter lumen 28 proximal to proximal end 54. Retrieval assembly 40 is positioned within everted balloon member such that retrieval assembly distal end 58 is proximal to catheter distal end 22. Retrieval assembly 40 also includes proximal end 60. Proximal end 60 of retrieval assembly 40 includes collapsible loop frame 61 coupled to mesh 41 and is coupled to distal end 56 of balloon member 30 at joint assembly 62. Joint assembly 62 also couples retrieval assembly 40 to flexible tether member 48.

FIG. 2B illustrates an alternate embodiment of the present invention showing thrombectomy system 110. Thrombectomy system 110 includes an elongate catheter 120 having delivery portion 122 and guidewire portion 124. Guidewire portion 124 having through lumen 126 extends proximally to the proximal end of catheter 120. Alternatively guidewire portion 124 and lumen 126 may extend proximally only a portion of the length of catheter 120 and have a configuration suitable for use as a “rapid exchange” system allowing the system 110 to reach a target site over a guidewire that has already been positioned at the target site. Delivery portion 122 includes lumen 128 extending from the proximal end to the distal end of catheter 120. Balloon member 130 is everted and positioned within lumen 128 of catheter delivery portion 122. Positioned within everted balloon member 130 in a collapsed configuration is retrieval assembly 140. Securing member 152, shown as a wound filament, affixes balloon member proximal end 154 to the distal end of delivery portion 122. Balloon member proximal end 154 and the distal end of delivery portion 122 may be secured using other means such as heat fusing, ultrasonic welding, multifilament winds and or gluing to insure a good bond and seal. The distal end 156 of balloon member 130 is completed sealed using any of the aforementioned techniques and positioned everted within catheter lumen 128 proximal to proximal end 154. Retrieval assembly 140 positioned adjacent distal end 156 of everted balloon member 130 includes distal portion 158 and proximal portion 160.

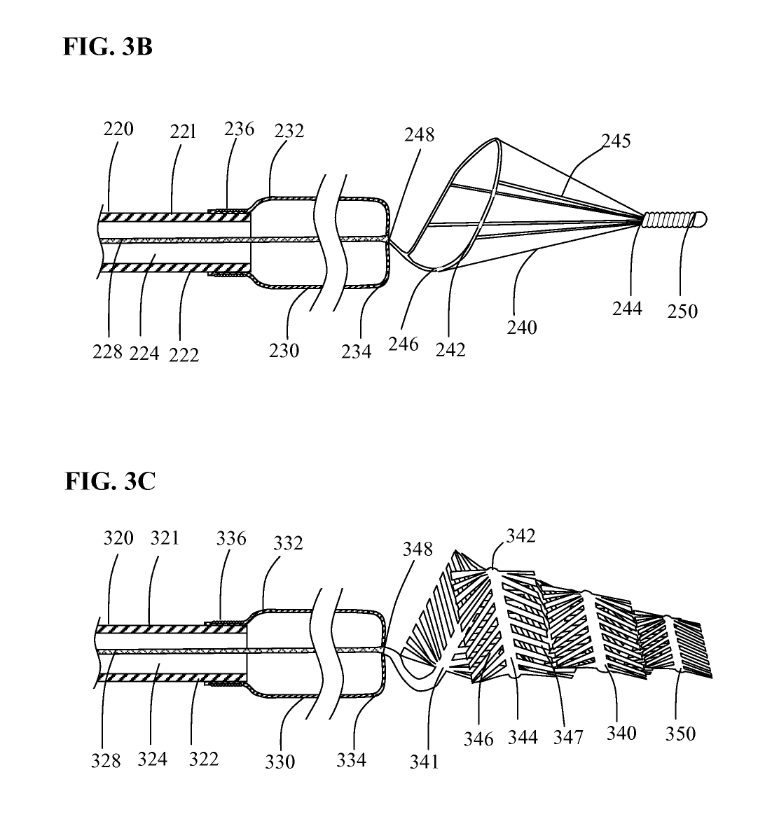

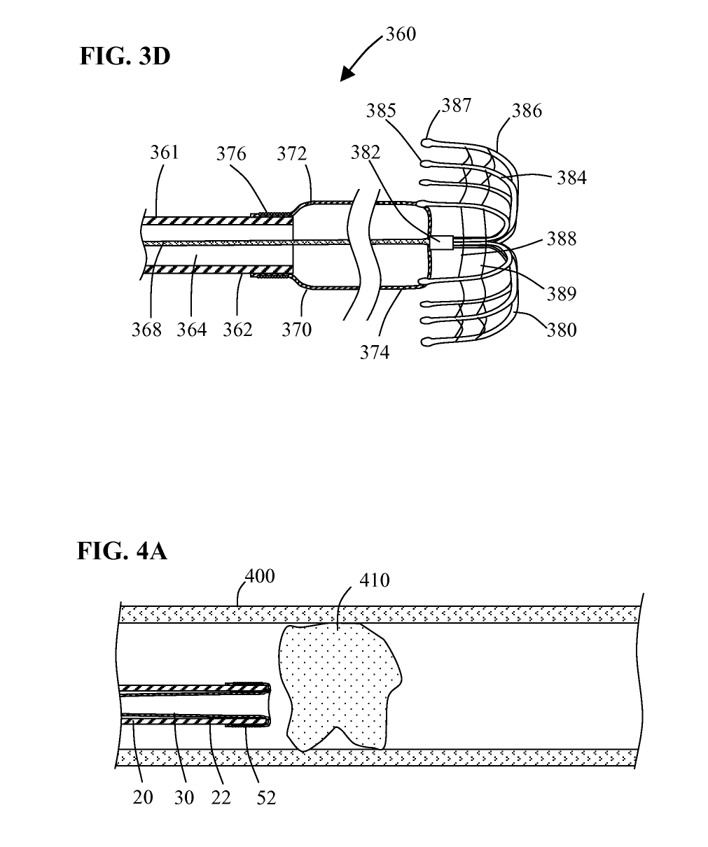

FIG. 3A shows an enlarged partial cross sectional view of the distal portion of thrombectomy system 10. Balloon member 30 is shown in an inflated configuration longitudinally extending distal to catheter distal end 22. In this configuration, balloon member distal end 56 is positioned distal to balloon member proximal end 54. The diameter of balloon member 30 may range from about 0.25 to about 1.5 times the diameter of catheter 20 and have a preferred range from about 0.5 to 1.2 times the diameter of catheter 20. Balloon members are preferably formed from an elongate thin walled noncompliant material. Suitable polymeric materials include nylon or polyester tubes having a wall thickness from 0.0001 inches to 0.010 inches with a preferred range of about 0.0005 inches to 0.005 inches. Other suitable materials include metallic thin film alloys such as nitinol having a wall thickness in the range of about 0.0001 inches to about 0.001 inches. Balloon members typically have a length that is substantially longer than the collapsed retrieval assemblies and range from about 40 mm to about 500 mm with a preferred range of about 50 mm to 400 mm. Retrieval assembly 40 is shown in a deployed configuration where distal end 58 is positioned distal to balloon member proximal end 54 and loop frame 61 of proximal end 60 is expanded. Typically, the expanded diameter of loop frame 61 is slightly larger than the inner diameter of the vessel at a target site. This diameter allows coupled mesh 41 at proximal end 60 to be adjacent or in contact with the inner wall of the vessel at a target site. Mesh 41 is formed of flexible material and may take commonly known configurations such as woven and nonwoven fabrics, braids, perforated materials, webs and nets.

FIGS. 3B through 3D are partial cross sectional views of thrombectomy systems according to alternate embodiments of the present invention that illustrate an inflated extended balloon member and different configurations of deployed retrieval assemblies. FIG. 3B shows an enlarged partial cross sectional view of the distal portion of thrombectomy system 220. Balloon member 230 is shown in an inflated configuration longitudinally extending distal to catheter distal end 222. In this configuration, balloon member distal end 234 is positioned distal to balloon member proximal end 232. The diameter of balloon member 230 may range from about 0.25 to about 1.5 times the diameter of catheter 221 and have a preferred range from about 0.5 to 1.2 times the diameter of catheter 221. Retrieval assembly 240 is shown in a deployed configuration where distal end 244 is positioned distal to balloon member proximal end 238 and loop frame 246 of proximal end 242 is expanded. Joint assembly 248 couples retrieval assembly 240 to flexible tether member 228 and balloon member distal end 234. Retrieval assembly 240 also includes a plurality of frame members, represented by frame member 245 that are coupled to loop frame 246 and extend towards distal end 244. The frame members are generally spaced apart about loop frame 246 and converge distally forming a “cage like” structure adapted to receive and retain emboli and or thrombus. Typically, the expanded diameter of loop frame 246 is slightly larger than the inner diameter of the vessel at a target site. This diameter allows the coupled frame members at proximal end 242 to be adjacent or in contact with the inner wall of the vessel at a target site. While the frame members, including representative frame member 245 are shown having a generally straight shape extending from loop frame 246 to distal end 244, it should be understood that the frame members may have alternative shapes including helixes, zigzag, arcuate, sinusoids and combinations thereof Located at distal end 244 is an atraumatic marker tip 250. Marker tip 250 preferably takes the form of a beaded flexible coil however other forms may e suitable such as flexible filaments or tubes that incorporate radiopaque elements.

FIG. 3C shows an enlarged partial cross sectional view of the distal portion of thrombectomy system 320. Balloon member 330 is shown in an inflated configuration longitudinally extending distal to catheter distal end 322. In this configuration, balloon member distal end 334 is positioned distal to balloon member proximal end 332. The diameter of balloon member 330 may range from about 0.25 to about 1.5 times the diameter of catheter 321 and have a preferred range from about 0.5 to 1.2 times the diameter of catheter 321. Retrieval assembly 340 includes an elongate flexible resilient backbone 341 that takes the form of a spiral having multiple adjacent winds preferably tapering distally from proximal end 342. Representative wind 344 of elongated backbone 341 is shown having a plurality of side extension members represented by side extension members 346 and 347. Representative side extension members 346 and 347 are only fixedly connected to backbone 341 and generally extend from opposite sides of backbone 341 at wind 344 spanning at least a portion of the gap between adjacent winds of backbone 341 as the winds taper towards distal end 350. Side extension members on one wind of backbone 341 may be adjacent to or overlap the side extension members on an adjacent wind of backbone 341. For illustrative purposes, side extension members 346 and 347 are shown having a straight shape and positioned at an angle relative to backbone 341, however multiple shapes and configurations including sinusoids, zigzags, varying widths, arcuate, open loops and combinations are also contemplated. Retrieval assembly 340 is shown in a deployed configuration coupled at joint assembly 348 to balloon member distal end 334 and flexible tether member 328, where distal end 350 is positioned distal to balloon member proximal end 332 and expanded proximal end 342. Typically, the expanded diameter of proximal end 342 is slightly larger than the inner diameter of the vessel at a target site. This diameter allows side extension members coupled to backbone 341 at proximal end 342 to be adjacent or in contact with the inner wall of the vessel at a target site.

FIG. 3D shows an enlarged partial cross sectional view of the distal portion of thrombectomy system 360. Balloon member 370 is shown in an inflated configuration longitudinally extending distal to catheter distal end 362. In this configuration, balloon member distal end 374 is positioned distal to balloon member proximal end 372. The diameter of balloon member 370 may range from about 0.25 to about 1.5 times the diameter of catheter 361 and have a preferred range from about 0.5 to 1.2 times the diameter of catheter 361. Retrieval assembly 380 is coupled to balloon member distal end 374 and elongate flexible tether member 368 at joint assembly 382. Retrieval assembly 380 is shown in a deployed configuration and includes a plurality of arcuate or generally “U” shaped capture arm members represented by arm members 384 and 386. The capture arm members have a first end that is coupled to joint assembly 382 and a second end that is free. The second ends of the capture arm members are spaced apart from each other and distributed radially about the central axis of joint assembly 382. The second end of capture arm members may include rounded atraumatic tips such as tip portions 385 and 387 of representative capture arm members 384 and 386. Tip portions may include markers for visibility under fluoroscopy, magnetic resonance or other imaging modalities. Typically, the expanded diameter of retrieval assembly 380 is slightly larger than the inner diameter of the vessel at a target site. This diameter allows the capture arm members to be adjacent to or in contact with the inner wall of the vessel at a target site. To further enable the secure capture of emboli or thrombus, flexible filaments such as filaments 388 and 389 may be coupled to and span adjacent capture arm members forming a web like configuration. A plurality of flexible filaments may take commonly known configurations such as woven and nonwoven fabrics, braids and nets.

Preferably, the retrieval assemblies of embodiments of the present invention comprise a biocompatible resilient material. Suitable resilient materials for loop frames, frame members, backbones, side extension members and arcuate members include metal alloys such as nitinol, titanium, stainless steel. Additional suitable materials include polymers such as polyimides, polyamides, fluoropolymers, polyetheretherketone(PEEK) and shape memory polymers. These materials may be formed into desired shapes by a variety of methods which are appropriate to the materials be in utilized such as laser cutting, thermal heat treating, vacuum deposition, electro -deposition, vapor deposition, chemical etching, photo etching, electro etching, stamping, injection molding, casting or any combination thereof. In addition, the biased resiliency of these materials allow a retrieval assembly with a normally expanded configuration to have a collapsed, small diameter configuration when constrained within a delivery catheter suitable for delivery to a target site and upon being deployed at a target site return to its expanded configuration.

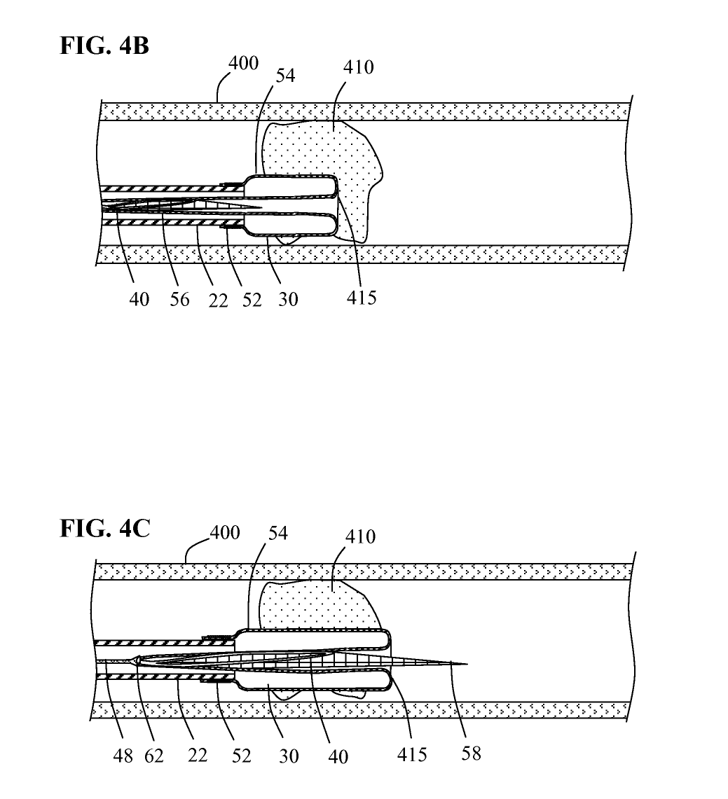

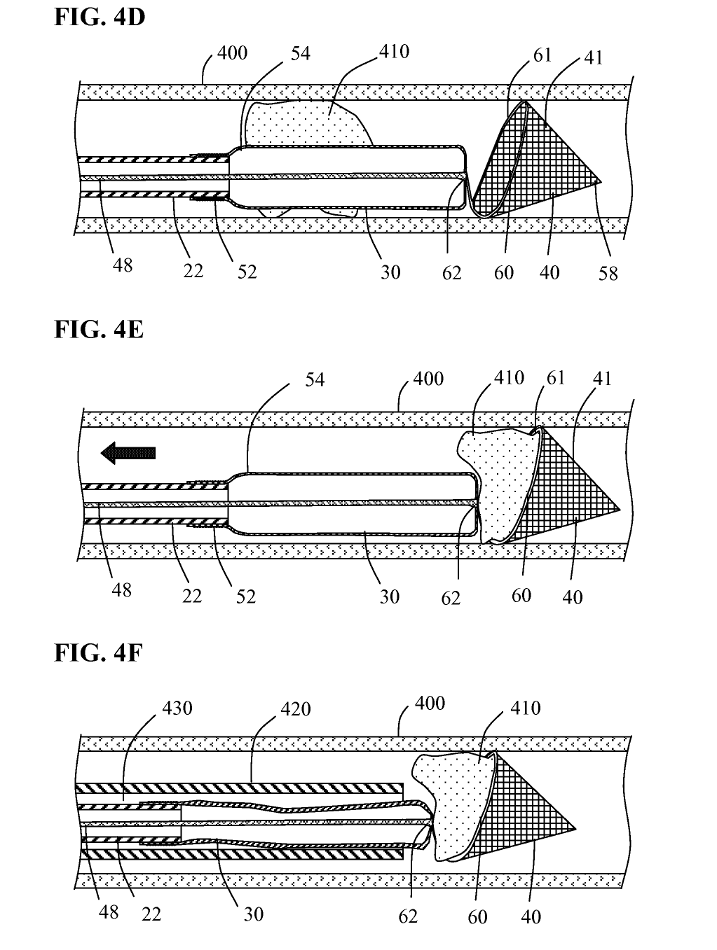

FIGS. 4A through 4F illustrate a method of deploying a retrieval assembly at a target site within a body lumen according to one embodiment of the present invention. The thrombectomy system 10 is positioned within a vessel 400. Catheter distal end 22 including extendable balloon member 30 are positioned at a target site adjacent to thrombus 410. An inflation source member (not shown) is coupled to the proximal end of the catheter 20 and used to apply fluid pressure to the lumen of catheter. The inflation source member preferably takes the form of a syringe (threaded or non-threaded), however other inflation sources such as a pressurized fluid source having a valve assembly or a controllable fluid delivery pump are also suitable. The preferred fluids include liquids such as saline and radiopaque contrasts solutions however gases such as carbon dioxide gas may be suitable for some system configurations. As the applied fluid pressure increases to a sufficient level, balloon member 30 begins to extend longitudinally from the catheter lumen. As balloon member 30 extends longitudinally from catheter 20, leading edge 415 of the balloon member 30 may encounter thrombus 410. While shown as large particle, thrombus 410 may have a varied composition that could comprise organized clot, tissue, plaque soft clot or even foreign objects. Dependant somewhat upon the size and composition of thrombus 410 balloon member 30 may extend longitudinally through a soft and compliant thrombus or between the inner vessel wall and a more rigid thrombus. Leading edge 415 of balloon member 30 is well suited to extend longitudinally between the more rigid thrombus and the vessel wall without perforating the vessel. With continued application of fluid pressure, balloon member 30 continues to extend longitudinally until retrieval assembly 40 is deployed and positioned distal to thrombus 410. Once retrieval assembly 40 is appropriately deployed, application of additional fluid pressure is unnecessary. In the deployed configuration, proximal end 60 of retrieval assembly 40 is expanded to preferably contact the inner wall of vessel 400. As shown in FIG. 4E, thrombectomy system 10 including tether member 48 is pulled proximally causing proximal end 60 of retrieval assembly 40 to engage the distal side of thrombus 410. Tether member 48 ensures that sufficient retraction force is applied directly to retrieval assembly 40 to capture thrombus 410. Thrombectomy system 10 with captured thrombus 410 may then be removed from the body. Alternatively, balloon member 30 may be deflated and thrombectomy system 10 with captured thrombus 410 pulled proximally within larger catheter 420 having lumen 430. Suction may then be applied to lumen 430 thus aiding retention of thrombus 410 during removal or to fragment and remove thrombus 410 from retrieval assembly 40.

Novel devices, systems and methods have been disclosed to perform vascular reconstruction and revascularization procedures within a mammal. Although preferred embodiments of the invention have been described, it should be understood that various modifications including the substitution of elements or components which perform substantially the same function in the same way to achieve substantially the same result may be made by those skilled in the art without departing from the scope of the claims which follow.

What is claimed is:

1. An emboli removal system to capture and remove emboli comprising:
an elongate tubular flexible member having proximal and distal ends and a lumen extending therethrough;
a longitudinally extendable member having proximal and distal ends, said proximal end of said extendable member being secured to the distal end of said flexible member and the distal end of said extendable member being everted and positioned within the lumen of said flexible member proximal to the proximal end of said extendable member;
an elongate flexible tether member slidably positioned within the lumen of said flexible member and having proximal and distal ends wherein the distal end of said tether member is coupled to the distal end of said extendable member; and
an emboli capture device having a first configuration that is collapsed and a first position within the lumen at the distal end of said flexible member and a second configuration that is expanded and a second position distal to the distal end of said flexible member, said emboli capture device being coupled to the distal end of said extendable member and operable between said first and second positions such that upon the application of fluid pressure said extendable member extends distally from said lumen thereby moving said emboli capture device from said first position within said lumen to said second position distal to the distal end of said flexible member.

2. The emboli capture system of claim 1 wherein said emboli capture device comprises a resilient material, said emboli capture device being resiliently biased to move from said first configuration to said second configuration.

3. The emboli removal system of claim 1 wherein said extendable member comprises a balloon.

4. The emboli removal system of claim 1 wherein said extendable member comprises a metallic thin film.

5. The emboli removal system of claim 1 wherein said extendable member comprises a non-compliant balloon.

6. The emboli removal system of claim 1 wherein said emboli capture device comprises a mesh.

7. The emboli removal system of claim 1 wherein said emboli capture device comprises a framework.

8. The emboli removal system of claim 1 wherein said emboli capture device comprises a primary member having a coil configuration.

9. The emboli removal system of claim 1 wherein said emboli capture device comprises a plurality of arcuate struts.

10. A method of removing emboli comprising the steps of:
positioning an emboli removal system including a catheter having an everted balloon and an emboli capture device positioned within the catheter lumen at its distal end within a body lumen adjacent emboli at a target site;
applying fluid pressure to said catheter lumen to extend said balloon and emboli capture device distally;
deploying said emboli capture device adjacent to the emboli at the target site;
capturing emboli within said emboli capture device;
removing said emboli from said target site.

11. The method according to claim 10 further comprising the step of repositioning said emboli removal system proximally while deploying said emboli capture device.

12. The method according to claim 10 further comprising the step of controlling the volume of fluid delivered to said balloon.

13. The method according to claim 10 further comprising the step of controlling the flow rate of fluid delivered to said balloon.

14. The method according to claim 10 wherein the step of deploying said emboli capture device further comprises deploying said emboli capture device distal to the emboli at the target site.

15. The method according to claim 10 wherein the step of capturing emboli within said emboli capture device comprises retracting said emboli capture device proximally to engage said emboli.

16. The method according to claim 10 wherein the step of removing said emboli from said target site comprises retracting said emboli capture device proximally to a larger lumen catheter.

17. The method according to claim 10 wherein the step of removing said emboli from said target site comprises retracting said emboli capture device proximally to a larger lumen catheter incorporating suction to remove said emboli.

18. An emboli removal system to capture and remove emboli comprising:
an elongate tubular flexible member having proximal and distal ends and a lumen extending therethrough;
an elongate longitudinally extendable balloon member having proximal and distal ends, said proximal end of said extendable balloon member being secured to the distal end of said flexible member and the distal end of said extendable balloon member being inverted and positioned within the lumen of said flexible member proximal to the proximal end of said extendable balloon member;
an elongate flexible tether member slidably positioned within the lumen of said flexible member and having proximal and distal ends wherein the distal end of said tether member is coupled to the distal end of said extendable balloon member; and
an emboli capture device having a first configuration that is collapsed and positioned within the inverted extendable balloon member at the distal end of said flexible member and a second configuration that is expanded and positioned distal to the distal end of said flexible member, said emboli capture device being coupled to the distal end of said extendable balloon member and operable between said first and second configurations such that upon the application of fluid pressure said extendable balloon member extends distally from said lumen thereby moving said emboli capture device from said first configuration within the inverted extendable balloon member to said second configuration distal to the distal end of said flexible member.

19. The emboli removal system of claim 18 wherein said emboli capture device comprises a shape memory material.

20. The emboli removal system of claim 18 wherein said emboli capture device comprises a framework.
